(19) United States
(12) Patent Application Publication
(10) Pub. No.: US 2025/0144136 A1
(43) Pub. Date: May 8, 2025

(54) FETAL MACROPHAGE AND CELL PREPARATION

(71) Applicant: KEIO UNIVERSITY, Tokyo (JP)

(72) Inventors: Shigeki SAKAI, Tokyo (JP); Kazuo KISHI, Tokyo (JP)

(73) Assignee: KEIO UNIVERSITY, Tokyo (JP)

(21) Appl. No.: 18/835,446

(22) PCT Filed: Feb. 9, 2023
(86) PCT No.: PCT/JP2023/004391

§ 371 Date: Aug. 2, 2024

(30) Foreign Application Priority Data
Feb. 10, 2022 (JP) 2022-019887

(51) Int. Cl. A61K 35/15; A61K 8/98; A61P 11/00; A61P 17/00; A61P 17/02; A61Q 19/00

(52) U.S. Cl. CPC A61K 35/15; A61K 8/982; A61P 11/00; A61P 17/00; A61P 17/02; A61Q 19/00

(57) ABSTRACT

A cell population including macrophages that are CD11b positive, F4/80 positive, CD180 positive, and CD9 positive and that are derived from a tissue selected from the group consisting of placenta, umbilical cord, and amnion with a percentage of 75% or more, the cell population having cell viability of 80% or more.

### TECHNICAL FIELD

This disclosure relates to a method for producing a cell population comprising fetal macrophages and a cell preparation comprising fetal macrophages.

### BACKGROUND ART

In the research of tissue regeneration and wound repair, stem cells, which are pluripotent or multipotent progenitor cells, are of interest because of their ability to generate a variety of mature human cell lineages. Patent Literature 1 and 2 (PTL 1 and 2) describe some examples using placenta-derived stem cells. These prior art documents aim at transplantation of adherent stem cells. However, there is a question in that it is unclear what state the cells having a therapeutic effect are in and it is also unclear whether the effect is a direct effect or a paracrine effect, as differentiation proceeds in the case of stem cells.

### CITATION LIST

### Patent Literature

[PTL 1] Japanese Patent Publication No. 2013-099343
[PTL 2] Japanese Patent Publication No. 2013-208135

### SUMMARY OF INVENTION

### Technical Problem

In the phenomenon of complete skin regeneration in mouse fetuses, which has long been studied in laboratories, the inventors have found cells in the placenta, umbilical cord, or amnion that are equivalent to fetal macrophages that suppress fibrosis and regenerate tissue.

The subject matter of the present disclosure is to provide a simple method for producing a cell population comprising fetal macrophages from placenta, umbilical cord, and amnion; a cell population comprising macrophages derived from a tissue selected from the group consisting of placenta, umbilical cord, and amnion; and a cellular therapeutic preparation including a cell population obtained by such production method or placenta-derived macrophages.

### Solution to Problem

The present disclosure encompasses the following embodiments.

Item 1. A cell population comprising macrophages that are CD11 b positive, F4/80 positive, CD180 positive, and CD9 positive and that are derived from a tissue selected from the group consisting of placenta, umbilical cord, and amnion with a percentage of 75% or more, the cell population having cell viability of 80% or more.

Item 2. The cell population according to Item 1, wherein the macrophages are non-adherent cells.

Item 3. The cell population according to Item 1, wherein the macrophages include Hofbauer cells.

Item 4. A cellular therapeutic preparation comprising as an active ingredient the cell population according to Item 1 or macrophages that are CD11 b positive, F4/80 positive, CD180 positive, and CD9 positive, and derived from a tissue selected from the group consisting of placenta, umbilical cord, and amnion.

Item 5. The cellular therapeutic preparation according to Item 4, wherein the cellular therapeutic preparation is an injectable preparation.

Item 6. The cellular therapeutic preparation according to Item 4 or 5, wherein the cellular therapeutic preparation is a therapeutic preparation for the treatment of a disease selected from the group consisting of an inflammatory disease, an immunomodulatory disease, and an autoimmune disease.

Item 7. The cellular therapeutic preparation according to Item 6, wherein the inflammatory disease includes inflammatory bowel disease or chronic inflammatory disease.

Item 8. The cellular therapeutic preparation according to Item 6, wherein the immunomodulatory disease includes a graft-versus-host disease, systemic lupus erythematosus, liver cirrhosis, pulmonary fibrosis, atopic dermatitis, or aging.

Item 9. The cellular therapeutic preparation according to Item 4 or 5, wherein the cellular therapeutic preparation is a therapeutic preparation for promotion of epithelialization, promotion of angiogenesis, inhibition of scarring, inhibition of thickening of the skin, suppression of wrinkle, wound healing, or suppression of fibrosis.

Item 10. A cosmetic composition comprising the cell population according to Item 1 or macrophages that are CD11 b positive, F4/80 positive, CD180 positive, and CD9 positive, and that are derived from a tissue selected from the group consisting of placenta, umbilical cord, and amnion.

Item 11. A macrophage derived from the differentiation of a pluripotent stem cell, the macrophage being CD11 b positive, F4/80 positive, CD180 positive, and CD9 positive.

Item 12. A method for producing a cell population including fetal macrophages comprising:
enzymatically treating a tissue selected from the group consisting of placenta, umbilical cord, and amnion with collagenase;
passing the enzymatically treated tissue through a mesh to separate a cell-containing fluid from fibrous connective tissue; and
purifying the cell population after passing the mesh to obtain a cell population including macrophages that are CD11 b positive, F4/80 positive, CD180 positive, and CD9 positive, the purifying step including
   contacting the cell population after removal of red blood cells with an anti-F4/80 antibody to fractionate the cell population having a relatively strong intensity of anti-F4/80 antibody; and/or
   purifying the cell population after removal of red blood cells with an anti-CD180 antibody and/or an anti-CD9 antibody.

Item 13. The method according to Item 12, wherein the percentage of macrophages that are CD11 b positive, F4/80 positive, CD180 positive, and CD9 positive in the cell population including fetal macrophages is 75% or more.

Item 14. The method according to Item 13, wherein the cell viability in the cell population including fetal macrophages is 80% or more.

Item 15. The method according to any one of Items 12 to 14, further comprising cryopreserving the cell population.

Item 16. The method according to any one of Items 12 to 14, further comprising culturing the cell population.

### BRIEF DESCRIPTION OF DRAWINGS

FIG. 1 is a schematic diagram illustrating development, differentiation, and migration of macrophages into tissues during an embryonic period.

FIG. 2 is measurement of the number of CD11 b-positive and F4/80-positive cells derived from skin on E13 and E18 by flow cytometry.

FIG. 3 is a wound healing process in mouse fetus.

FIG. 4 is measurement of the number of cells derived from placenta ((A) and (D)), amnion ((B) and (E)), and umbilical cord ((C) and (F)) by flow cytometry. (A)-(C) detection of the number of CD180-positive cells, (D)-(F) detection of the number of CD9-positive cells.

FIG. 5 is micrographs of mouse fetal back skins. Left: E18-Skin macrophage administration group on embryonic day 18. Center: Control PBS group. Right: E13-Skin macrophage administration group on embryonic day 13.

FIG. 6 is graphs indicating (A) number of blood vessels in each group and (B) number of cells with αSMA-positive myofibroblasts in each group.

FIG. 7 is photographs of skins of adult mice implanted with fetal macrophages on day 4 after wound formation. (A) and (C): control PBS-treated group, (B) and (D): fetal macrophage administration group.

FIG. 8 is an analysis of cells including fetal macrophages isolated from E18 placenta, frozen, thawed, and cultured. (A) measurement of CD180 positive cells by flow cytometry, (B) measurement of CD9 positive cells by flow cytometry, and (C) a micrograph of cells floating in the supernatant.

FIG. 9 is histologically stained photographs of sections of scar tissues. (A) a representative example of the group (n=3) in which PBS was injected intravenously as a control for the scar. (B) right: a representative example of the group (n=3) in which macrophages derived from the placenta on embryonic day 18 were injected.

FIG. 10 is a change in skin wounds over time in the control group (n=6) injected with PBS via tail vein (left) and in the group (n=6) injected with placenta-derived macrophages (E18-placenta macrophages) on embryonic day 18.

FIG. 11 is graphs showing wound contraction over time for each of the control group and E18-Placenta macrophage group, with the wound area on the first day regarded as 100%. *T test P<0.05.

FIG. 12 is photographs of histological staining of skin on the fourth day after wound formation. (A) a photograph of histological staining of skin of the mouse in the control group, (B) a magnified photograph of the area enclosed by the left frame of (A), (C) a magnified photograph of the area enclosed by the right frame of (A), (D) a photograph of histological staining of skin of the E18-Placenta macrophage group, (E) a magnified photograph of the area enclosed by the left frame of (D), and (F) a magnified photograph of the area enclosed by the right frame of (D).

FIG. 13 is graphs of the average epithelialization rate of the control group and the E18-Placenta macrophage group on day 4 after wound formation.

FIG. 14 is (A) a photograph of the wound site of the mouse in the control group on day 14 after wound formation, (B) a photograph of histological staining of skin of the mouse in the control group on day 14, (C) a magnified photograph of (B), (D) a photograph of the wound site of the mouse in the E18-Placenta macrophage group on day 14 after wound formation, (E) a photograph of histological staining of skin in the E18-Placenta macrophage group on day 14, and (F) a magnified photograph of (E).

FIG. 15 is a change in skin wounds over time in the control group injected with PBS via tail vein (n=2, two wounds on each side in each mouse) (left) and in the group injected with FACS fractionated placenta-derived macrophages (FACS E18-Placenta macrophages) (right) on embryonic day 18 (n=3, two wounds on each side in each mouse) (right).

FIG. 16 is (A) a photograph of the wound site of the mouse in the control group on day 14 after wound formation, (B) a photograph of histological staining of skin of the mouse in the control group on day 14, (C) a photograph of the wound site of the mouse in the FACS E18-Placenta macrophage group on day 14 after wound formation, and (D) a photograph of histological staining of skin of E18-Placenta macrophage group on day 14.

FIG. 17 is measurements of mean values of inflammatory cytokines and aging markers in the skin of each group of mice (n=4). (A) NF-kB, (B) IL-6, (C) IL-1α and (D) TGFβ. E18-Placenta: mice treated with placenta-derived fetal macrophages; PBS: mice treated with PBS. *P<0.05.

FIG. 18 is (A) a photograph of histological staining of PBS-treated group, (representative example out of n=4) (B) a photograph of histological staining of the group administered with placenta-derived fetal macrophages (representative example out of n=4), in pulmonary fibrosis models.

FIG. 19 is measurements of inflammatory cytokines and aging markers in the skin of each group of mice (n=3). (A) NF-kB, (B) IL-1α, (C) p16ink4a and (D) CEBPB. Young: young mice, Old: old mice, YSMp: old mice treated with yolk sac macrophages, PlaMp: old mice treated with placenta-derived fetal macrophages.

FIG. 20 is measurements of inflammatory cytokines and markers of aging in the liver of each group of mice (n=3). (A) NF-kB, (B) IL-1α, (C) p16ink4a and (D) CEBPB. Young: young mice, Old: old mice, YSMp: old mice treated with yolk sac macrophages, PlaMp: old mice treated with placenta-derived fetal macrophages.

FIG. 21 is measurements of inflammatory cytokines and markers of aging in the lungs of each group of mice (n=3). (A) NF-kB, (B) IL-1α, (C) p16ink4a, and (D) CEBPB. Young: young mice, Old: old mice, YSMp: old mice treated with yolk sac macrophages, PlaMp: old mice treated with placenta-derived fetal macrophages.

FIG. 22 is (A) gross observation of scratching in ADMJ mice, a mouse model of atopic dermatitis. (Left) intravenous administration of PBS four times, (Right) intravenous administration of E18-Placenta macrophages four times. (B) photographs of histological staining of ADMJ mice. (Left) intravenous administration of PBS five times, (Right) intravenous administration of E18-Placenta macrophage five times. The bar is 250 μm.

FIG. 23 is examples of significant efficacy in additional cases. (Left) a photograph of the face of an ADMJ mouse before treatment, (Right) a photograph of the face of the same mouse after administration of E18-Placenta macrophages.

FIG. 24 is mean values of scratch in each group in ELAN analysis (n=3 per group). NT: no treatment (PBS); E18-MP administration: E18-Placenta macrophage administration.

FIG. 25 is (A) FACS analysis of E18-Placenta macrophages cultured in DMEM low glucose medium supplemented with heat-inactivated 10% BSA and 1% PS and seeded in the adherent 6-well plate. (B) FACS analysis of the R9 region in (A). (C) FACS analysis of E18-Placenta macrophages cultured in DMEM/F12 medium supplemented with heat-inactivated 10% BSA and 1% PS and seeded in the adherent 6-well plate. (D) FACS analysis of the R9 region in (C).

FIG. 26 is (A) FACS analysis of E18-Placenta macrophages cultured in DMEM low glucose medium supplemented with heat-inactivated 10% BSA and 1% PS and seeded in the adherent 6-well plate. (B) FACS analysis of the R9 region in (A). (C) FACS analysis of E18-Placenta macrophages cultured in DMEM low glucose medium supplemented with deactivated 10% BSA, 1% PS and CSF1 and seeded in the adherent 6-well plate. (D) FACS analysis of the R9 region in (C).

FIG. 27 is (A) FACS analysis of E18-Placenta macrophages cultured in DMEM low glucose medium supplemented with heat-inactivated 10% BSA, 1% PS and seeded in the non-adherent 6-well plate. (B) FACS analysis of the R9 region in (A). (C) FACS analysis of E18-Placenta macrophages cultured in low glucose medium supplemented with heat-inactivated 10% BSA, 1% PS, and CSF1 and seeded in the non-adherent 6-well plate. (D) FACS analysis of the R9 region in (C).

FIG. 28 is an expression of each of various marker molecules in (A) mice adult spleen and (B) E-18-Placenta macrophages. For each sample, (a) CD45, (b) F4/80, (c) CD9, and (d) counts of MHC class 2, (e) MHC class II in R6, and (f) MHC class II in R7.

### EMBODIMENTS FOR CARRYING OUT THE INVENTION

A singular form herein covers both the singular and the plural unless otherwise expressly stated herein or clearly contradicted by context. In this specification, “comprise” is a concept that also includes “consisting essentially of” and “consisting of only.”

“Treatment” herein refers to the treatment of a subject having one or more diseases, which is a condition or a symptom caused by the condition, and means to ameliorate, alleviate, or eliminate the condition or the symptom. “Prevention” means the treatment of a subject who is at risk of developing a disease but does not currently have the condition or the symptom.

“Cell population” herein refers to a group of cells that include two or more cells. The cell population may contain one type of cell, or two or more types of cells.

“Fetal macrophage” herein refers to a macrophage derived from the yolk sac or a macrophage differentiated from a macrophage derived from the yolk sac.

“Adult macrophages” herein refers to a macrophage derived from a fetal liver monocyte or a macrophage differentiated from a macrophage derived from a fetal liver monocyte.

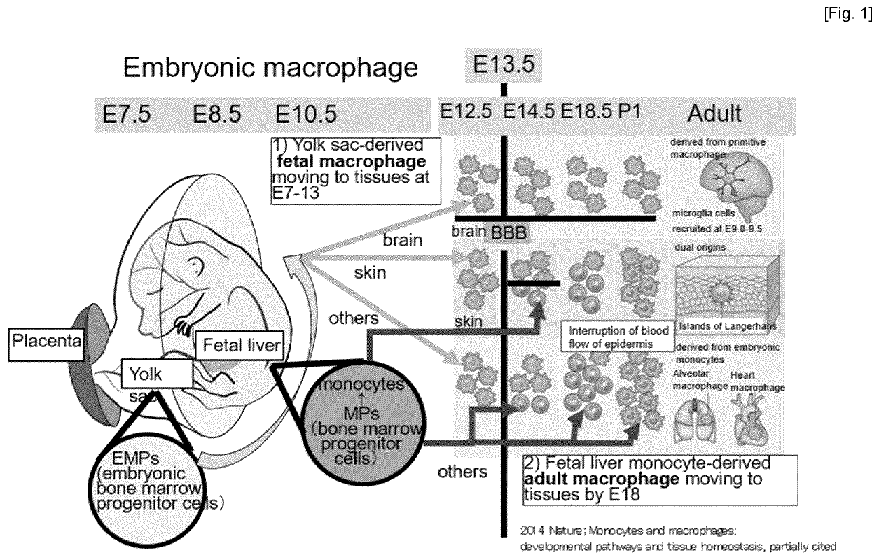

Monocytes and macrophages are involved in tissue homeostasis and immunity. As shown in FIG. 1, among macrophages arising in the embryonic stage, yolk sac-derived macrophages are macrophages that arise from primitive hematopoietic progenitor cells in the blood islands of the yolk sac of fetal mouse embryos and that migrate to tissues during embryonic period E7-13. Fetal embryonic liver monocyte-derived macrophages are macrophages that are differentiated from monocytes derived from hematopoietic cells derived from the fetal liver and that migrate to tissues by E18 after the yolk sac-derived macrophages are settled. Recently, studies in mice have established that tissue-endemic macrophages in the brain are yolk sac-derived macrophages and that tissue-endemic macrophages in the skin include two types: macrophages derived from the yolk sac and macrophages derived from fetal liver monocytes (Nature Reviews Immunology, volume 14, pages 392-404 (2014)).

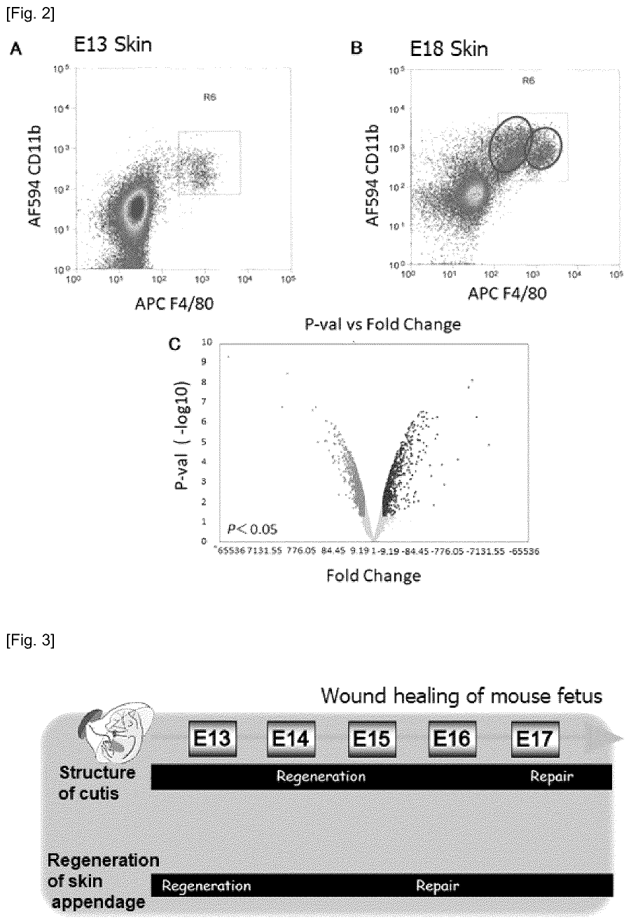

The inventors examined the cells in the skin of E13 mouse fetuses and E18 mouse fetuses by flow cytometry using an anti-mouse/human CD11 b antibody (BioLegend) and an anti-mouse F4/80 antibody (BioLegend), a specific marker for macrophages. We confirmed that only one type of macrophage (i.e., fetal macrophages) was present in E13 skin, while two types of macrophages (i.e., fetal macrophages and adult-type macrophages) were present in E18 skin (FIG. 2).

As shown in FIG. 3, during the process of wound healing in mouse fetuses, the dermis regenerates after wounding from embryonic stage E13 to E16, but wounds can only be repaired and remain as scars after E17. The borderline between regeneration and repair in the skin appendages is E13.5. This is believed to be due to a change in the type of macrophages in the skin.

The inventors hypothesized that when fetal macrophages are abundant, skin wounds would be healed in a regenerative fashion. If we can isolate fetal macrophages, we will be able to transplant cells that control an inflammatory reaction and that direct tissues toward regeneration, rather than stem cell transplants, which are transplants of tissue-building cells, and we will be able to treat various diseases, which could not be achieved previously.

The inventors found that cells equivalent to fetal macrophages, which are specific to mouse fetuses and regenerate tissues, also exist in the placenta, umbilical cord, and amnion, and succeeded in isolating and identifying them. These fetal macrophages derived from tissues such as placenta have an action to treat or alleviate various diseases including fibrotic diseases. The transplantation of the fetal macrophages, which are tissue-resident macrophages, from outside the body is a unique invention, because once depleted, they cannot be supplemented.

According to some aspects of the present disclosure, there is provided a cell population comprising macrophages that are CD11b positive, F4/80 positive, CD180 positive, and CD9 positive, and that are derived from a tissue selected from the group consisting of placenta, umbilical cord, and amnion with a percentage of 75% or more, the cell population having cell viability of 80% or more.

The percentage of CD11 b-positive, F4/80-positive, CD180-positive, and CD9-positive cells in the cell population is the percentage of the number of the cell population that are CD11 b-positive, F4/80-positive, CD180-positive, and CD9-positive of the total number of cells. The CD11 b-positive, F4/80-positive, CD180-positive, and CD9-positive cells particularly refer to fetal macrophages.

A tissue is a mammalian tissue, and a mammal includes, but is not limited to, a human, a mouse, a rat, a pig, and a cow. A human and a mouse are preferred.

In some embodiments, CD11b positive, F4/80 positive, CD180 positive, and CD9 positive cells are CD163 negative.

In some embodiments, the percentage of CD11 b-positive, F4/80-positive, CD180-positive, and CD9-positive cells in the above cell population is 80% or more, preferably 90% or more.

In some embodiments, the cell viability in the above cell population is 80%, preferably 90% or more. The cell viability in the cell population refers to the percentage of the number of viable cells out of the total number of cells in the cell population.

In some embodiments, the macrophages are non-adherent cells. Therefore, the macrophages of the present disclosure and the cell populations containing them have less risk (e.g., pulmonary emboli) that can occur due to cell adhesion, as seen with adherent cells, and are advantageous for intravenous administration.

In some embodiments, the macrophages include Hofbauer cells. Hofbauer cells are a type of macrophage found in the placenta by Dr. J. Isfred Isidore Hofbauer. Hofbauer cells express CD11 b and F4/80 (Front Immunol. 2017; 8: 888, Immunol Rev. 2014 November; 262(1): 36-55, Am J Reprod Immunol. 2011 October; 66(4): 336-348). Even if the fetal macrophages are not Hofbauer cells themselves, if an invention satisfies the requirements of the

s, it is included in the technical scope of the invention.

The cell population including the above macrophages, or the macrophages that are that are CD11 b positive, F4/80 positive, CD180 positive, and CD9 positive and that are derived from a tissue selected from the group consisting of placenta, umbilical cord, and amnion as described above, may be used as an active ingredient of medicinal drugs or quasi-drugs to treat various diseases or as an ingredient of cosmetic products to improve skin condition(s).

According to some aspects of the present disclosure, there is provided a cellular therapeutic preparation comprising as an active ingredient the above-mentioned cell population or the macrophages that are CD11 b positive, F4/80 positive, CD180 positive, and CD9 positive, and that are derived from a tissue selected from the group consisting of placenta, umbilical cord, and amnion. The term “cellular therapeutic preparation” may be substituted for “pharmaceutical composition.”

The cellular therapeutic preparation may include a pharmacological carrier other than the cell population of the present disclosure, or other than the macrophages that are CD11 b positive, F4/80 positive, CD180 positive, and CD9 positive and that are derived from a tissue selected from the group consisting of placenta, umbilical cord, and amnion as described above. The “pharmacological carrier” may be any organic or inorganic carrier material customarily used as an ingredient in pharmaceutical preparations. In solid preparations, the pharmacological carrier may be formulated as an excipient, a binder, a disintegrant, a lubricant, a coating agent, and the like. In liquid preparations, the pharmacological carrier may be formulated as a solvent, a dissolution aid, a suspending agent, an isotonic agent, a pH adjuster, a buffer, a painkiller, and the like.

In addition to the above cell populations or the above macrophages that are CD11 b positive, F4/80 positive, CD180 positive, and CD9 positive and that are derived from a tissue selected from the group consisting of placenta, umbilical cord, and amnion, the cellular therapeutic preparation may include a drug. The term “drug” may include one or more other agents administered to a subject for the purpose of treating a disease.

The cellular therapeutic preparation may also include a surfactant, a preservative, a stabilizer, a painkiller, a local anesthetic, a preservative, a wetting agent, an emulsifier, a dispersing agent, and a dissolution aid to the extent that they do not interfere with the effect of the invention.

In some embodiments, there is provided a method of treating a disease of a subject or a method of transplanting cells into a subject, comprising administering a therapeutically effective amount of the above cellular therapeutic preparation to the subject.

The subject, also referred to as a patient or a test subject, is preferably a mammal. The mammal includes, but is not limited to, a human, a mouse, a rat, a pig, and cattle, with a human and a mouse being preferred, and a human being most preferred].

The disease to be treated by the cellular therapeutic preparation includes, but is not limited to, a disease selected from the group consisting of an inflammatory disease, an immunomodulatory disease, and an autoimmune disease.

The inflammatory disease includes, but is limited to, inflammatory bowel disease and chronic inflammatory disease.

The immunomodulatory diseases includes, but is not limited to, a graft-versus-host disease, systemic lupus erythematosus, liver cirrhosis, pulmonary fibrosis, atopic dermatitis, and aging.

In particular, the cell preparation currently used for fibrotic diseases is human MSCs (mesenchymal stem cells), and there are few cellular therapies specific to fibrosis. MSCs are adhesive cells and easy to culture, but because of their adhesive nature, there is a great risk of pulmonary embolization and other problems with intravenous administration. The use of the non-adherent fetal macrophages of the present disclosure reduces this risk.

The cellular therapeutic preparation can also be used as a therapeutic preparation for promotion of epithelialization, promotion of angiogenesis, inhibition of scarring, inhibition of thickening of the skin, suppression of wrinkle, wound healing, or suppression of fibrosis.

A method of administration of the cellular therapeutic preparation includes, but is not limited to, subcutaneous injection, intralymphatic injection, intravenous injection, intraperitoneal injection, intratumoral injection, or direct local injection or direct local implantation.

The cellular therapeutic preparation of the present invention can be used as an injectable preparation, or a preparation for transplantation as a cellular mass or a sheet-like structure. In some embodiments of the present disclosure, the fetal macrophages derived from a tissue selected from the group consisting of placenta, umbilical cord, and amnion are suitable as an injectable because they are non-adherent cells.

The dosage of the cellular therapeutic preparation is the amount of cells that, when administered to a subject, can cause a therapeutic effect on the disease compared to a subject not receiving the cellular therapeutic preparation. The specific dosage can be determined according to the form of administration, method of administration, purpose of use, and age, weight, and symptom of the subject, or the like. As an example, 10⁵ to 10⁹ cells/kg body weight per administration in a human (e.g., adult) is preferred, and 10⁵ to 10⁸ cells/kg body weight is more preferred, in terms of the number of fetal macrophage cells.

The frequency of administration, the interval between doses, and the dosing period can be determined as appropriate. For example, the frequency of administration may include, but is not limited to, once a day, one to three times a week, and once every two to four weeks. The interval between doses may be, but is not limited to, 3 days or more to 1 month. The dosing period may be one time only, or multiple times within a period of one week to six months.

According to some aspects of the present disclosure, there is provided a cosmetic composition comprising the above cell population or the macrophages that are CD11 b positive, F4/80 positive, CD180 positive, and CD9 positive, and that are derived from a tissue selected from the group consisting of placenta, umbilical cord, and amnion.

The cosmetic composition may include, in addition to the above-mentioned cell population or the above-mentioned macrophages are CD11 b positive, F4/80 positive, CD180 positive, and CD9 positive, one or more ingredients used in topical agents and cosmetics to the extent that they do not impair the effect of the invention. Such ingredients include water, an alcohol, an oil and a fat, a surfactant, an excipient, an emulsifier, an isotonic agent, a buffer, a diluent, a dissolution aid, a preservative, a stabilizer, an antioxidant, a gelling agent, powders, a water-soluble polymer, a UV protective agent, an inclusion compound, a fragrance, a salt, a pH adjuster, an animal or microbial extract, a plant extract, a blood circulation stimulant, an astringent, a whitening agent, an anti-inflammatory agent, an active oxygen scavenger, a cell activator, a moisturizer, a chelating agent, a keratinolytic agent, an enzyme, a hormone, a vitamin, or the like.

The cosmetic compositions may be used as a composition for inhibiting or improving inflammation, a composition for promoting epithelialization, a composition for inhibiting or improving wrinkles, or a composition for inhibiting or improving skin aging.

The subject to which the cosmetic composition is applied is a mammal, preferably a human.

The cosmetic composition of the present disclosure is preferably a topical skin care preparation or a basic cosmetic. Such topical skin care preparation and cosmetic may include, but are not particularly limited to, water-based emulsions, oil-in-water emulsions, oil-in-water emulsions, and multi-layered emulsions. The forms of the topical skin care preparation and the cosmetic can be any form such as powder, liquid, emulsion, paste, cream, gel, mousse, ointment, sheet, or the like.

Instead of the macrophages that are CD11 b positive, F4/80 positive, CD180 positive, and CD9 positive and that are derived from a tissue selected from the group consisting of placenta, umbilical cord, and amnion, the macrophages that are CD11 b positive, F4/80 positive, CD180 positive, and CD9 positive may be produced by differentiation of pluripotent stem cells. Such macrophages derived from differentiation of pluripotent stem cells are preferably macrophages derived from differentiation of pluripotent stem cells in vitro. Various methods are known for inducing differentiation of pluripotent stem cells into macrophages (e.g., Higaki K. et al., Mol Ther Nucleic Acids, 12: 793-804, doi:10.1016/j.omtn.2018.07.017 (2018), and Irina Lyadova et al., Cell & Bioscience, volume 12, Article number: 96 (2022)). A skilled person can produce such macrophages by inducing differentiation of pluripotent stem cell with ordinary skill. Whether or not the cell population of the present disclosure has been obtained can be confirmed by the expression of CD antigen(s) or the F4/80 antigen.

“Pluripotent stem cells” are stem cells that are pluripotent, capable of differentiating into many types of cells that exist in the body, and also capable of self-proliferation. Examples of pluripotent stem cells include embryonic stem (ES) cells, embryonic stem (ntES) cells derived from cloned embryos obtained by nuclear transfer, embryonic germ cells (EG cells), induced pluripotent stem (iPS) cells, and multipotent cells derived from cultured fibroblasts or bone marrow stem cells (Muse cells). Pluripotent stem cells are preferably mammalian pluripotent stem cells, and more preferably human pluripotent stem cells, such as human ES cells or human iPS cells.

A cellular therapeutic preparation and a cosmetic composition including the macrophages derived from differentiation of pluripotent stem cells that are CD11 b positive, F4/80 positive, CD180 positive, and CD9 positive are also included within the scope of the invention. The details of the cellular therapeutic preparation and the cosmetic composition are described above.

Next, a method for producing the above-mentioned cell population will be described.

According to some aspects of the present disclosure, there is provided a method for producing a cell population including fetal macrophages comprising: enzymatically treating a tissue selected from the group consisting of placenta, umbilical cord, and amnion with collagenase; passing the enzymatically treated tissue through a mesh to separate a cell-containing fluid from fibrous connective tissue; and purifying the cell population after passing the through the mesh.

The tissue is a mammalian tissue. The mammal includes, but is not limited to, a human, a mouse, a rat, a pig, and a bovine, with a human and a mouse being preferred.

The collected tissue is homogenated using scissors or the like. The tissue may be stored in a culture medium without shredding. For example, the tissue can be stored in a culture medium at 2-8° C. for 24-48 hours. The enzymatic treatment of the tissue with collagenase is performed by immersing the tissue in a solution containing collagenase, and shaking or stirring. Commercially available media can be used for the solution, and DMEM medium is the preferred medium in terms of selectively obtaining fetal macrophages. A preferred solution is DMEM medium containing collagenase Type 1 and albumin at a concentration of 0.1 w/v % to 5 w/v %. The concentration of collagenase is preferably 0.05 w/v % to 0.2 w/v %. The temperature of the enzymatic treatment is preferably 30 to 40° C. Stirring can be performed by stirring at 700-800 rpm for at least 30 minutes using a stirring device. The upper limit of the period for enzymatic treatment is not particularly limited; however, since the prolonged enzymatic treatment may change the properties of the cells, it can generally be performed within 3 hours, preferably within 90 minutes, e.g., within 60 minutes.

After stirring, the tissue is separated into a fibrous connective tissue and a cell-containing liquid. A mesh-size cell strainer is set in a sterile tube to filter the cell-containing liquid after the tissue digestion by natural dropping, so that the fibrous connective tissue remains on the mesh and only the cellular component passes through the mesh. The mesh size is preferably 40-200 μm, more preferably 40-150 μm, and more preferably 70-150 μm. By setting the mesh size (pore size of the mesh) in the above range, a decrease in cell viability can be prevented by letting the cells drop naturally, without applying pressure.

After passing through the mesh, a hemolysis buffer is added to the cell-containing liquid after the filtration to remove red blood cell components, the liquid is allowed to stand at room temperature before centrifugation is performed. The centrifugation condition is preferably 200-500×g for 5 minutes. In this way, fetal macrophages are collected.

According to the above production method, it is not necessary to perform the enzymatic treatment multiple times as in the past, and even a single enzymatic treatment can easily and aseptically produce isolated fetal macrophages derived from a tissue selected from the group consisting of placenta, umbilical cord, and amnion. Even when multiple enzymatic treatments are conducted, decrease in the recovery rate due to repeated centrifugation and washing during the process can be prevented. Therefore, a large amount of fetal macrophages that are derived from a tissue selected from the group consisting of placenta, umbilical cord, and amnion can be prepared easily in a short time.

Furthermore, as is the case with any cell preparation, it is preferable that the production steps be as simple as possible in order to eliminate bacterial, viral, or other contamination. According to the above production method, repeated centrifugation and washing during the process can be omitted, thus eliminating contamination by microorganisms or the like as much as possible. The hemolytic buffer is added to the cell-containing liquid separated from the fibrous connective tissue and the liquid is centrifuged to remove red blood cells.

The step of purifying the cell population after passing through the mesh (or after removing red blood cells, if the red blood cells are removed) can be performed using a substance that can specifically recognize fetal macrophages, for example, a substance that can recognize a membrane surface antigen of fetal macrophages. For example, the F4/80 antigen is strongly expressed on the fetal macrophages and weakly expressed on the adult macrophages; the CD180 antigen is expressed on the fetal macrophages but not on the adult macrophages; and the CD9 antigen is also expressed on the fetal macrophages but not on the adult macrophages. Therefore, by purifying a cell population containing fetal macrophages with the anti-F4/80 antibody, the anti-CD180 antibody, the anti-CD9 antibody, or a combination of two or three of them, the fetal macrophages can be purified to a higher purity.

When a plurality of antibodies is used in the purification step, the timing of mixing each antibody with the cell population may be the same or different.

In some embodiments, the cell population after removal of red blood cells is contacted with an anti-F4/80 antibody, and the cell population in which the intensity of the anti-F4/80 antibody is relatively stronger than other cell populations is sorted by cell sorting (cell fractionation) to obtain D11b-positive, F4/80-positive, CD180-positive, and CD9-positive fetal macrophages. The fractionation of the cell population in which the intensity of the anti-F4/80 antibody is relatively stronger than other cell populations refers to fractionation of the cell population in which the intensity of the anti-F4/80 antibody is relatively stronger, especially the cell population in which the intensity of the anti-F4/80 antibody is strongest, from among a plurality of cell populations.

In some embodiments, the cell population including fetal macrophages are purified with an anti-CD180 antibody and an anti-CD9 antibody.

In some embodiments, the cell population including fetal macrophages are purified with an anti-F4/80 antibody, an anti-CD180 antibody, and an anti-CD9 antibody. In some embodiments, the cell population including fetal macrophages are purified with an anti-F4/80 antibody, an anti-CDb11b antibody, an anti-CD180 antibody, and an anti-CD9 antibody.

In some embodiments, the fetal macrophages are non-adherent cells. The fetal macrophages remain floating even after culturing in an appropriate medium for a period of time (e.g., one week in DMEM medium supplemented with 10% fetal bovine serum). Thus, the fetal macrophages isolated or produced by the production method of the present disclosure have less risk of cell adhesion (e.g., pulmonary embolism), as seen with adherent cells, and are advantageous for intravenous administration.

In some embodiments, the percentage of CD11 b-positive, F4/80-positive, CD180-positive, and CD9-positive cells in the cell population obtained by the above production method is preferably 50% or greater, more preferably 75% or greater, more preferably 80% or greater, and more preferably 90% or greater. The CD11 b-positive, F4/80-positive, CD180-positive, and CD9-positive cells are preferably macrophages derived from a tissue selected from the group consisting of placenta, umbilical cord, and amnion.

In some embodiments, the cell viability in the cell population obtained by the above production method is preferably 50% or greater, more preferably 60% or greater, more preferably 70% or greater, more preferably 80% or greater, and more preferably 90% or greater.

The cell viability in the cell population refers to the percentage of the number of viable cells out of the total number of cells in the cell population.

In some embodiments, the percentage of CD11 b-positive, F4/80-positive, CD180-positive, and CD9-positive cells in the cell population is 75% or more, and the cell viability in the cell population is 80% or more.

In some embodiments, the method for producing a cell population including fetal macrophages may further comprise the step of cryopreserving the cell population. The cell population is preferably mixed with a cryopreservation solution containing serum prior to the cryopreservation step so that the cell population is preserved at a high viability. The cryopreservation solution may be manufactured according to published literature, or a commercial product may be used. The cryopreservation includes, for example, lowering the temperature in a programed freezer at a freezing rate of −1 to −2° C./min and cryopreserving in a deep freezer at −80° C. to −100° C. Alternatively, the cell population may be cryopreserved in a liquid nitrogen tank. If frozen, the cryopreservation solution containing the frozen cell population can be thawed in a hot water bath. The temperature of the hot water bath is preferably between 30-40° C., more preferably 37° C., but is not limited to this.

In some embodiments, the method for producing a cell population including fetal macrophages may further comprise the step of culturing the cell population. The process of culturing includes, for example, diluting the collected cells in DMEM medium containing serum and culturing the cells in a culture vessel. The serum may be, for example, fetal bovine serum or human serum, and the serum concentration in the medium is preferably 5 w/v % or higher, and more preferably 10 w/v % or higher.

When the cell population including fetal macrophages are produced from a human sample, a different method from the above production method may be used since the amount of the sample is large. For example, as briefly explained, a tissue selected from the group consisting of placenta, umbilical cord, and amnion is cut, digested with trypsin, passed through a gauge to collect the cells that have passed through, and the collection is fractionated by density gradient using Percoll (trademark), Ficoll (trademark), or a reagent for cell separation that can separate macrophages from blood components. The fraction can be used as a cell population including fetal macrophages after collection. For methods of isolating cells from placenta, umbilical cord, and amnion, see Appios A. et al., (2021). Bio-protocol 11(11): e4044. doi: 10.21769/BioProtoc.4044, Am J Reprod Immunol. 2011 October; 66(4): 336-348. doi:10.1111/j.1600-0897.2011.01006. See also: 10.1111/j.1600-0897.2011.01006.x, for example.

Optionally, trypsin digestion may be followed by washing with a buffer such as PBS and further digestion with collagenase. Also, optionally, the residue that did not pass through the gauge may be further digested with collagenase V and DNase I before passing through the gauge and pooling with the previous cell collection. The fetal macrophages can be sorted by FACS (fluorescence-activated cell sorting system) or the like.

Examples are given below to illustrate the present disclosure in more detail, however, the present disclosure is not limited to these examples.

The following examples are provided to further illustrate the disclosure.

### EXAMPLES

### Example 1: Expression and Identification of Fetal Macrophages and Adult Macrophages in Skin

ICR mice on day 13 of pregnancy were sedated under isoflurane inhalation anesthesia, and sacrificed to death by dislocation of the cervical vertebra. After disinfection with 70% ethanol, the skin and peritoneum were incised with scissors, and the uterus including the fetus was removed and placed in a sterile petri dish. The uterus was incised to collect fetuses. The collected fetuses were washed well with PBS. The right and left dorsal skins of each fetus were harvested without including a spine and auricular cartilage, under a microscope. The fetal skin was homogenated with scissors, collected in a sterile Eppendorf tube, diluted in DMEM with 0.1 w/v % collagenase Typ1 and 2 w/v % BSA, and stirred at 750 rpm for 45 min at 37° C. with a constant-temperature shaker-stirrer. A cell strainer with a mesh size of 100 μm was set in a 50 ml centrifuge tube and the collected cells were poured into the centrifuge tube. The cell population was collected by natural dropping, leaving a fibrous connective tissue on the mesh. A hemolytic buffer was administered to the collected cell population and allowed to stand at room temperature for 10 min. The mixture was centrifuged at 300×g for 5 min at 4° C. for separation to generate a pellet of cells.

A blocking buffer supplemented with the anti-CD16/32 antibody as a background reducing antibody for flow cytometry was added on ice and mixed at 4° C. for 15 minutes. The anti-CD11 b antibody and the anti-F4/80 antibody were added to the mixture and incubated for 30 minutes. To remove excess antibodies, 0.5 w/v % BSA 2 mM EDTA in PBS (pH 7.2) (hereafter referred to as a FACS buffer) was added and the mixture was centrifuged again at 300×g for 5 minutes at 4° C. for separation to generate a pellet of cells. After removing the supernatant, the FACS buffer was added again to the precipitated pellet and analyzed by flow cytometry.

A pellet of cells was also prepared from the back skin of ICR mouse fetuses on day 18 of pregnancy using the same method as the above cell isolation method, except that the stirring condition was at 37° C. at 750 rpm for 60 minutes using a constant-temperature shaker-stirrer. After removing the supernatant, the FACS buffer was added again and analyzed by flow cytometry.

Flow cytometry was performed using fluorescence activated cell sorting (FACS). FACS is an instrument that can quantitatively measure the amount of antigen on the cell surface by staining cells with one or more fluorescent antibodies, placing the cells in a liquid stream, and passing the cells through the focus of a laser beam to measure the fluorescence emitted by individual cells. In addition to analysis, only specific cells can be isolated based on the results. The fluorescent antibodies used in this study are shown below.
TruStain fcX™ (anti-mouse CD16/32) (BioLegend) Alexa Fluor (registered trademark) 594
anti-mouse/human CD11 b (BioLegend) APC anti-mouse F4/80 (BioLegend)
7-AAD Viability Staining Solution (BioLegend)

### (Results)

As shown in FIG. 2A, a CD11 b-positive F4/80 strongly positive cell population was blotted from fetal skin on embryonic day 13. This cell population was enclosed by a square gate and fetal macrophages were collected by cell sorting.

As shown in FIG. 2B, macrophages were also collected from fetal skin on embryonic day 18 by cell sorting after the cell population was enclosed by a square gate.

Of the two cell populations enclosed in FIG. 2B, the CD11 b-positive F4/80 strongly positive cell population corresponds to fetal macrophages, and the CD11b-positive F4/80 weakly positive cell population corresponds to adult-type macrophages.

The cells isolated in the above experiments were collected in 1 ml of ISOGEN, RNA was extracted, and cDNA was synthesized. The amount of each cDNA was equally diluted and microarray expression analysis was performed by Clariom S (Thermo Fisher Scientific) (FIG. 2C). FIG. 2C shows P-val on the vertical axis and fold change on the horizontal axis. Comparing the macrophages collected from fetus skin on embryonic day 13 (enclosed by a square gate) and those collected fetus skin on embryonic 18-day-old fetus skin (enclosed by a square gate), more than 2-fold changes in expression levels of about 2000 genes were observed.

### Example 2: Expression and Identification of Fetal Macrophage in Placenta, Umbilical Cord, and Amnion

ICR mice on day 18 of pregnancy were sedated under isoflurane inhalation anesthesia, sacrificed to death by dislocation of the cervical vertebra. After disinfection with 70% ethanol, the skin and peritoneum were incised with scissors, uterus including the fetus was removed and placed in a sterile petri dish. The uterus is incised to collect the placenta, umbilical cord, and amnion. The harvested tissues were washed well with PBS, and then the placenta, umbilical cord, and amnion were transferred into separate Eppendorf tubes. The tissues were homogenated with scissors, diluted in DMEM with 0.1 w/v % collagenase and Typ1.2 w/v % BSA, and stirred at 750 rpm for 60 min at 37° C. with a constant-temperature shaker-stirrer. A cell strainer with a mesh size of 100 μm was set in a 50 ml centrifuge tube and the collected cells were poured into the centrifuge tube. The cell population was collected by natural dropping, leaving a fibrous connective tissue on the mesh. A hemolytic buffer was administered to the collected cell population and allowed to stand at room temperature for 10 min. The mixture was centrifuged at 300×g for 5 min at 4° C. for separation to generate a pellet of cells. A blocking buffer supplemented with the anti-CD16/32 antibody was added on ice and mixed at 4° C. for 15 minutes. The anti-CD11 b antibody, the anti-F4/80 antibody, the anti-CD180 antibody, the anti-CD9 antibody, and 7AAD were added to the mixture and incubated for 30 minutes. To remove excess antibodies, 0.5 w/v % BSA 2 mM EDTA in PBS (pH 7.2) (hereafter referred to as FACS buffer) was added, and the cells were centrifuged again at 300×g for 5 minutes at 4° C. for separation to make a pellet of cells. After removing the supernatant, the FACS buffer was added again and analyzed using flow cytometry.

### (Results)

The fetal macrophages are CD45-positive, CD11 b-positive, F4/80 strong signal (i.e., high expression level), and CD180-positive. CD9 is positive, but its expression level decreases with development.

The adult macrophages are CD45-positive, CD11 b-positive, F4/80 weak signal (i.e., low expression level), CD180-negative, and CD9-negative.

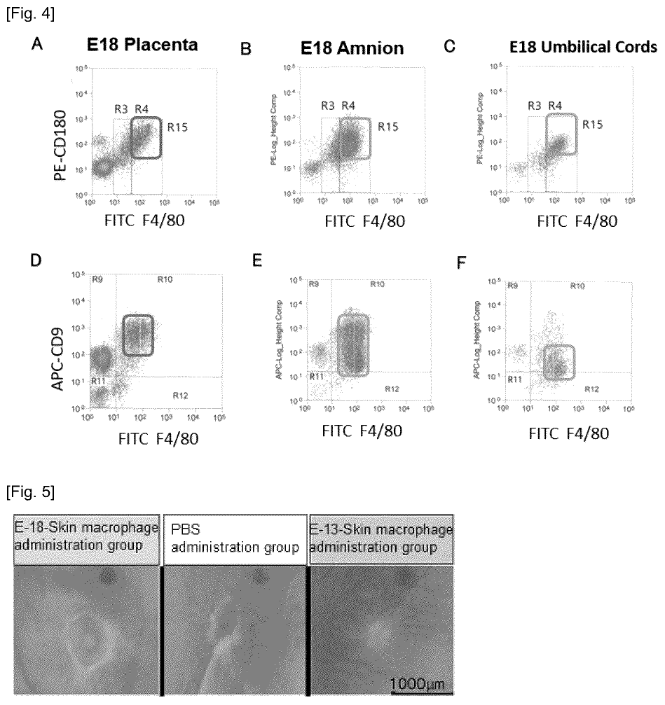

As shown in FIG. 4(A), in the placenta-derived pellet, the area enclosed by the square is the cell population corresponding to fetal macrophages, and the percentage of CD45+ (+ is positive, − is negative, and the same applies below) and F4/80+ cells out of 100% of the total cells was 27% and the percentage of CD180+ cells out of 100% of the total cells was 5.87%.

As shown in FIG. 4(B), in the amnion-derived pellet, the percentage of CD45+ and F4/80+ cells out of 100% of the total cells was 22.2% and the percentage of CD180+ cells out of 100% of the total cells was 14.7%.

As shown in FIG. 4(C), in the umbilical cord-derived pellet, the percentage of CD45+ and F4/80+ cells out of 100% of the total cells was 5.99%, and the percentage of CD180+ cells out of 100% of the total cells was 3.42%.

As shown in FIG. 4(D), the area enclosed by the square is the cell population corresponding to fetal macrophages, and the number of CD9+ cells was almost equal to the number of CD180+ cells.

As shown in FIG. 4(E), the number of CD9+ cells was half that of CD180+ cells.

As shown in FIG. 4(F), the number of CD9+ cells was 30% of the number of CD180+ cells.

The results of FIGS. 4(D)-(F) indicate that the placenta, umbilical cord, and amnion of mice on pregnancy day 18 all contained fetal macrophages. Since the placenta is considered to have the largest tissue volume and the highest content of fetal macrophages among the placenta, umbilical cord, and amnion, the placenta was used in the following experiments.

### Example 3: Healing Effect of Fetal Macrophages on Wounds of Mouse Fetuses

BL6 mice on pregnancy days 13 and 18 were treated under the same conditions as in Example 1, from removal of fetuses, collection of back skins, enzymatic treatment, separation with mesh, administration of a hemolytic buffer, and subsequent centrifugation to generate pellets of cells. However, the stirring conditions with the constant-temperature shaker-stirrer were 37° C., 750 rpm for 45 minutes for the skins on embryonic day 13, and 37° C., 750 rpm for 60 minutes for the skins on embryonic day 18.

A blocking buffer supplemented with the anti-CD16/32 antibody was added on ice, and mixed at 4° C. for 15 minutes. The Anti-F4/80 antibody with magnetic beads was added to the mixture and incubated for 15 minutes. To remove excess antibodies, the FACS buffer was added and the mixture was centrifuged again at 300×g for 5 minutes at 4° C. for separation to create a pellet of cells. After removing the supernatant, the FACS buffer was added again and MACS (Magnetically Activated Cell Sorting) was performed to collect the cell population. The cell population was centrifuged at 300×g for 5 minutes at 4° C. for separation to generate a pellet of cells. The cell population of the cell pellet collected from fetal skin on day 13 was referred to as E13-Skin macrophages and the cell population of the cell pellet collected from fetal skin on day 18 was referred to as E18-Skin macrophages.

After disinfection, the skin and peritoneum were incised with scissors to expose the uterus. The uterus was incised under the microscope and a small hole was made in the amnion. Full-layer skin defect wounds were created in the back skin of the fetus with a 1 mm punch. (1) E13-Skin macrophages, (2) PBS, and (3) E18-Skin macrophages were administered subcutaneously at the wounds using capillary tubes. After staining the wounds with a drop of the Dil reagent, the amnion was sutured and closed with 9-0 nylon, and the abdomen was closed with 4-0 nylon after intraperitoneal administration of ritodrine hydrochloride. After disinfection with 70% ethanol, the skin and peritoneum were incised with scissors and the fetuses were removed. The collected fetuses were washed well with PBS, positioned under a fluorescent stereomicroscope, and the wounds were photographed.

### (Results)

The results of the experiment showed that the wounds of embryonic E14.5 mice were dramatically inhibited from scarring and the skin regenerated without scars when the E13-Skin macrophage macrophages were administered (FIG. 5, right), whereas the scarring of the wounds was confirmed when the E18-Skin macrophages were administered (FIG. 5, left).

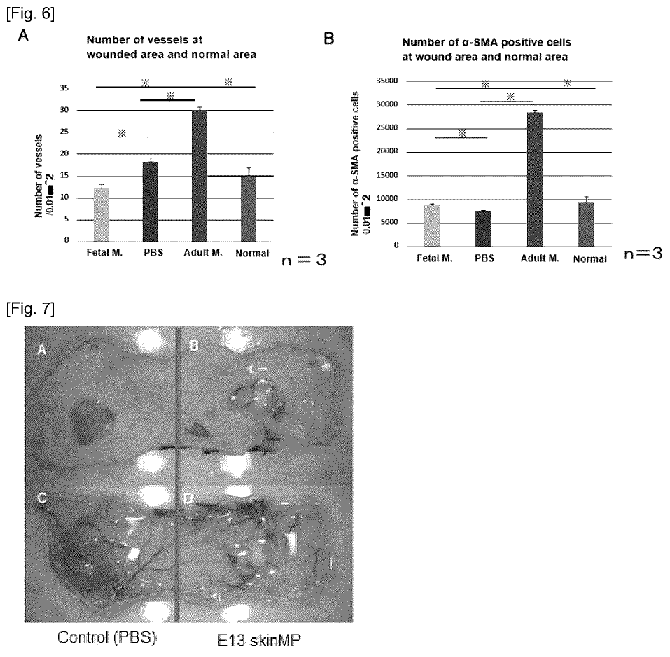

The E13-Skin macrophage administration group also showed an increase in the number of blood vessels, suggesting that the wounds were undergoing repair (FIG. 6(A)). The αSMA-positive myofibroblasts were more numerous at the margins of the wounds in the E18-Skin macrophage administration group (FIG. 6(B)). In the E13-Skin macrophage administration group, almost the same regeneration as that of normal areas was observed. There was a significant difference from the PBS group. The effect of the fetal macrophages in E13-Skin macrophages on skin regeneration and the effect of the adult-type macrophages in E18-Skin macrophages on the repair by scarring were confirmed.

### Example 4: Healing Effect of Fetal Macrophages on Wounds in Adult Mice

A cell pellet of E13-Skin macrophages was obtained from BL6 mice at day 13 of pregnancy by the same method described in Example 3.

Three 8- to 10-week-old male BL6 mice were sedated under isoflurane inhalation anesthesia, dewormed, and two 5 mm circular wounds were made in the skin on the back side using a biopsy trepan. One of the wounds was injected with 0.5 ml of the collected E13-Skin macrophages suspended in PBS and the other wound was injected with 0.5 mL of PBS using a micro cannula.

On days 1, 4, 7, and 11 after wound formation, the condition of the wounds was photographed along with a caliper scale, and the gross findings were compared. The size of the each wound was also measured, and the shrinkage rate was calculated and represented on a graph.

The same experiment was performed again, and the wound and surrounding skin were cut out and soaked in 4% PFA four days after injection of the E13-Skin macrophages. Some specimens were embedded in paraffin and the tissues were observed.

### (Results)

The microscopic observation also showed that the group treated with the E13-Skin macrophages, which are rich in fetal macrophages, showed more wound shrinkage than the group treated with PBS (FIGS. 7(A)-(D)). FIGS. 7(A) and 7(C) show the front and back of the same skin, and FIGS. 7(B) and 7(D) show the front and back of another skin from FIG. 7(A).

On day 4 after wound formation, the E13-Skin macrophage administration group showed an average of 46.9% wound shrinkage compared to day 1, while the PBS group showed an average of 57.1% shrinkage compared to day 1, indicating a higher contraction rate in the E13-Skin macrophage administration group.

In this example, the administration of fetal macrophages promoted epithelialization and angiogenesis in acute wounds of adult mice.

### Example 5: Culture of Placenta-derived Fetal Macrophages

ICR mice on day 18 of pregnancy were sedated under isoflurane inhalation anesthesia, and sacrificed to death by dislocation of the cervical vertebra. After disinfection with 70% ethanol, the skin and peritoneum were incised with scissors. The uterus including the placenta was removed and placed in a sterile petri dish. The uterus was incised to harvest the placenta. The harvested placenta was washed well with PBS and collected in a sterile Eppendorf tube. The placenta was homogenated with scissors, diluted in DMEM with 0.1 w/v % collagenase Typ1 and 2 w/v % BSA, and stirred at 750 rpm for 60 minutes at 37° C. with a constant-temperature shaker-stirrer. A cell strainer with a mesh size of 100 μm was set in a 50 ml centrifuge tube and the collected cells were poured into the centrifuge tube. The cell population was collected by natural dropping, leaving a fibrous connective tissue on the mesh. A hemolytic buffer was administered to the collected cell population and allowed to stand at room temperature for 10 minutes. The mixture was centrifuged at 300×g for 5 minutes at 4° C. for separation to create a pellet of cells.

A blocking buffer supplemented with the anti-CD16/32 antibody was added on ice and mixed at 4° C. for 15 minutes. Then the anti-F4/80 antibody with magnetic beads was added and incubated for 15 minutes. To remove excess antibody, the FACS buffer was added, and the cells were centrifuged and separated again at 300×g for 5 minutes at 4° C. to generate a pellet of cells. After removing the supernatant, the FACS buffer was added again and MACS (Magnetically Activated Cell Sorting) was performed to collect the cell population. The cell pellet was centrifuged again at 300×g for 5 minutes at 4° C. for separation. The cell population of the cell pellet collected from the day 18 placenta was referred to as E18-Placenta macrophages.

Depending on the case, 1 ml of a cryopreservation solution CellBanker (registered trademark) was added to every 1×10⁵ to 1×10⁷ cell population collected. The cell suspension was aliquoted into cryotubes, the temperature was lowered at a freezing rate of −1 to −2° C./min in a programmed freezer, and the cell suspension was stored frozen at −80° C. in a deep freezer.

### (Results)

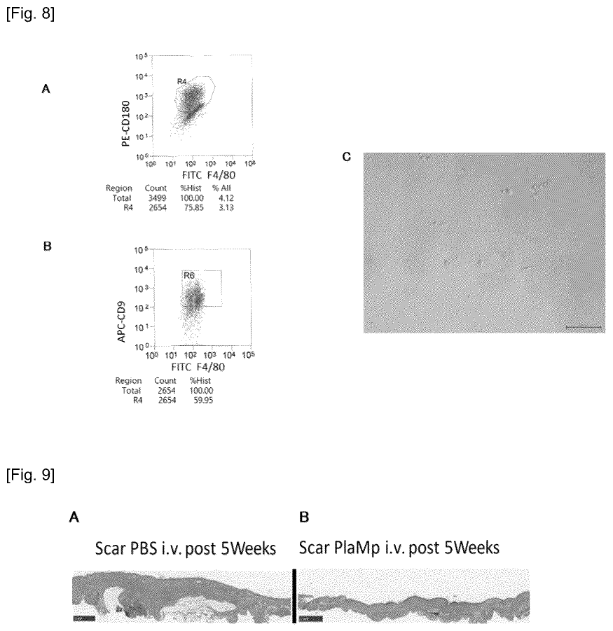

Most of the F4/80 positive cells in the E18-Placenta macrophages obtained by MACS were non-adherent cells and survived in DMEM supplemented with heat-inactivated 10% FBS and 1% PS. They retained their properties even after about 7 days of culture (FIGS. 8(A)-(C)). Strongly differentiated macrophages and fibroblasts adhered to the dish, and more vulnerable fetal macrophages could be collected as floating cells (it was confirmed by FACS that the collected non-adherent cells were fetal macrophages).

Since the fetal macrophages are non-adherent cells, it was expected that there would be little risk of pulmonary embolism, even when administered intravenously. In the following Examples 7 to 9, experiments of intravenous administration were conducted.

### Example 6: Culture and Purification of Placenta-derived Fetal Macrophages

1×10⁶ of the E18-Placenta macrophage cells collected in Example 5 were cultured in a 10 cm plastic dish. When the E18-Placenta macrophages were frozen in Example 5, they were thawed in a warm bath at 37° C. The thawed cells were diluted in DMEM supplemented with heat-inactivated 10% FBS and 1% PS, centrifuged at 1800 rpm for 3 min at 4° C., and the cell pellet was collected and cultured. After 3-7 days of culture, the supernatant was collected, and centrifuged at 300×g for 5 minutes at 4° C. for separation to generate a pellet of cells.

Then, the anti-CD11 b antibody, the anti-F4/80 antibody, the anti-CD180 antibody, the anti-CD9 antibody, and 7AAD were added and incubated for 30 minutes. To remove excess antibodies, the FACS buffer was added, and the cells were centrifuged again at 300×g for 5 minutes at 4° C. for separation to generate a pellet of cells. After removing the supernatant, the FACS buffer was added again and flow cytometry was performed to obtain a cell population rich in fetal macrophages.

In this way, the E18-Placenta macrophages can be further cultured, and fetal macrophages can be purified.

### Example 7: Inhibition of Skin Thickening in Skin Scar Tissue by Placenta-derived Fetal Macrophages

E18-Placenta macrophages were obtained from the placenta of GFP mice on day 18 of pregnancy under the same conditions as in Example 5.

Two groups of three 8- to 10-week-old male BL6 mice were sedated under isoflurane inhalation anesthesia, and a 2×2 cm full-layer skin defects were created on the dorsal skin. The defects were protected with a film to prevent bleeding, and the film was removed the next day. Four weeks after wound formation, a scarred hairless area on the back was identified. One of the wounds was injected with 0.1 ml of the E18-Placenta macrophages suspended in PBS and the other wound was injected with 0.1 ml of PBS through the femoral vein in the groin.

Five weeks after femoral vein injection, the animals were sedated under isoflurane inhalation anesthesia, and sacrificed to death by dislocation of the cervical vertebra. The skin scar tissue was photographed along with a caliper scale, and the surrounding skin was cut out and soaked in 4% PFA. Some specimens were embedded in paraffin and the tissues were observed.

In the skin scar tissue, intravenous administration of the E18-Placenta macrophages derived from the placenta on embryonic day 18 showed an anti-inflammatory effect and inhibited thickening and fibrosis of the epidermis and dermis (FIG. 9(B)). The inflammation of scar tissue and thickening and fibrosis of epidermis and dermis were observed in the PBS group (FIG. 9(A)).

### Example 8: Confirmation of the Healing Effect of Fetal Macrophages on Adult Mouse Wounds (MACS)

A pellet containing cells of the E18-Placenta macrophages was obtained from BL6 mice on day 18 of pregnancy by the method described in Example 5. Thereafter, the E18-Placenta macrophages were obtained by MACS in the same manner as in Example 5.

Six 8- to 10-week-old male BL6 mice were sedated under isoflurane inhalation anesthesia, dewormed, and two 8 mm circular wounds were made on the skin on the back side using a biopsy trepan. One of the wounds was injected with 0.1 ml of 1×10⁵ E18-Placenta macrophages suspended in PBS collected by MACS and the other wound was injected 0.1 ml of PBS through the tail vein using a micro cannula.

### (Results)

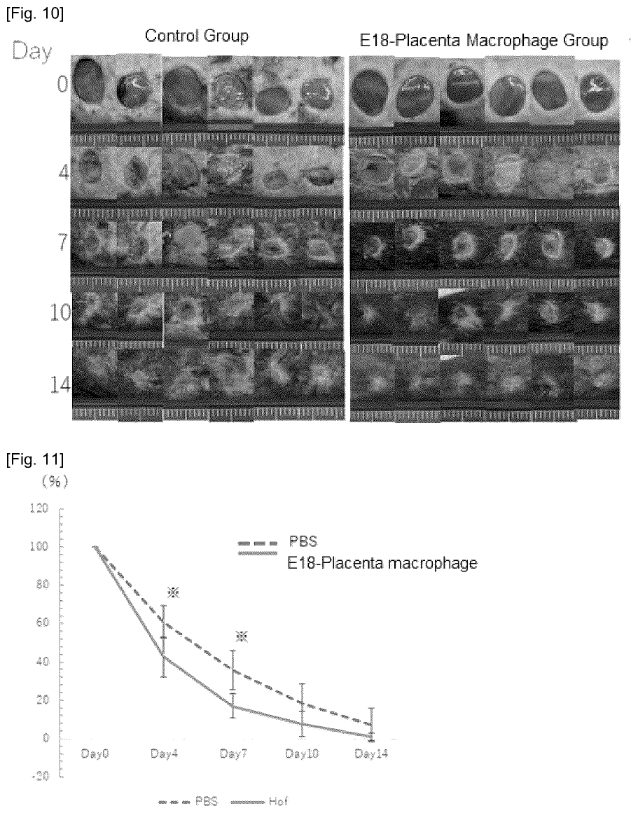

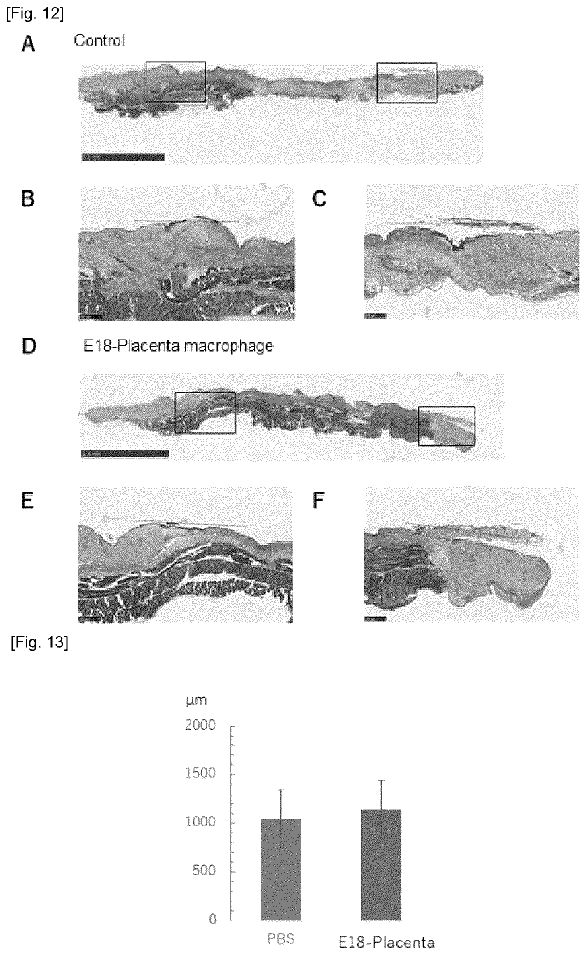

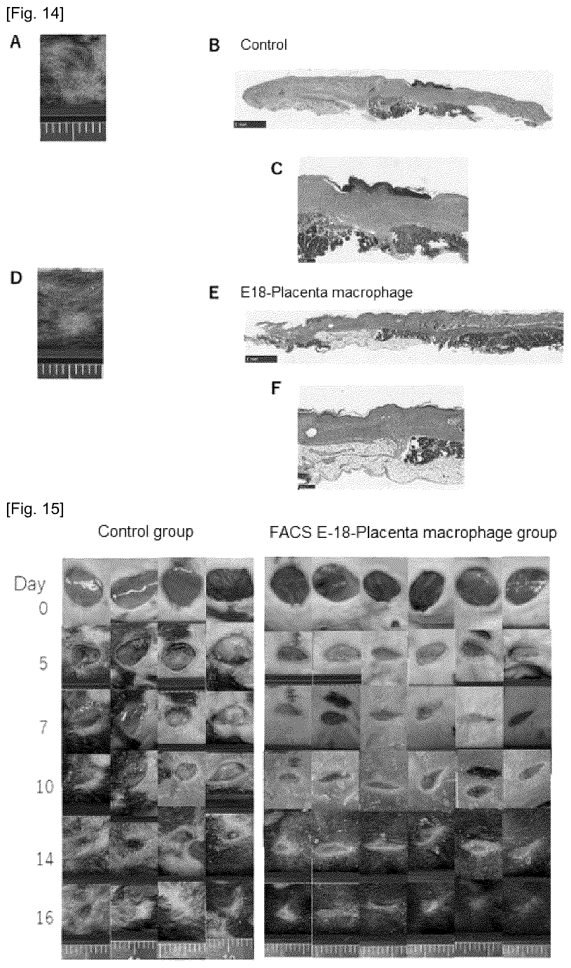

Gross findings of acute skin wounds showed early shrinkage by intravenous administration of the E18-Placenta macrophages (FIGS. 10 and 11). On day 4 of wound formation, a thick layer of granulation was observed in the PBS group (FIGS. 12(A)-(C)) while a thin layer of granulation was observed in the E18-Placenta macrophage group (FIGS. 12(D)-(F)). A comparison of epithelialization rates showed a trend toward higher epithelialization rates in the E18-Placenta macrophage group, but no significant difference (FIG. 13). 14 days later, the PBS group showed inflammation of scar tissue, and thickening and fibrosis of the epidermis and dermis (FIGS. 14(A)-(C)). The E18-Placenta macrophage group was able to suppress inflammatory cells as well as thickening and fibrosis of the epidermis and dermis (FIGS. 14(D)-(F)).

### Example 9: Confirmation of the Healing Effect of Fetal Macrophages on Adult Mouse Wounds (FACS)

A pellet containing cells of E18-Placenta macrophages was obtained from BL6 mice on day 18 of pregnancy by the method described in Example 5. The blocking buffer supplemented with the anti-CD16/32 antibody was added to the cell pellet on ice and mixed at 4° C. for 15 minutes. The anti-CD11b antibody, the anti-F4/80 antibody, the anti-CD180 antibody, the anti-CD9 antibody, and 7-AAD were then added and incubated at 4° C. for 30 minutes. To remove excess antibodies, the FACS buffer was added and the cells were centrifuged again at 300×g for 5 minutes at 4° C. for separation to create a pellet of cells. After removing the supernatant, the FACS buffer was added again to the precipitated pellet and the pellet was analyzed using flow cytometry to obtain E18-Placenta macrophages by FACS.

Five 8- to 10-week-old male BL6 mice were sedated under isoflurane inhalation anesthesia, dewormed, and two 8 mm circular wounds were made in the skin on the back side using a biopsy trepan. 3 mice were injected with 0.1 ml of 1×10⁵ E18-Placenta macrophages collected by FACS and suspended in PBS and the other two mice were injected with 0.1 ml of PBS through the tail vein using a micro cannula. The skin of each group was collected and half was fixed in 4% PFA for 24 hours before being cut into paraffin sections. The other half was homogenized in ISOGEN, after which RNA was extracted and cDNA synthesized. The synthesized cDNA was measured by real-time PCR using a TaqMan probe.

### (Results)

Gross findings of acute skin wounds showed early shrinkage by intravenous administration of the E18-Placenta macrophages fractionated by FACS (FIG. 15). On day 14, inflammation of scar tissue, and thickening and fibrosis of the epidermis and dermis were observed in the PBS group (FIGS. 16(A) and (B). The FACS E18-Placenta macrophage group was able to suppress inflammatory cells as well as thickening and fibrosis of the epidermis and dermis (FIGS. 16(C) and (D)).

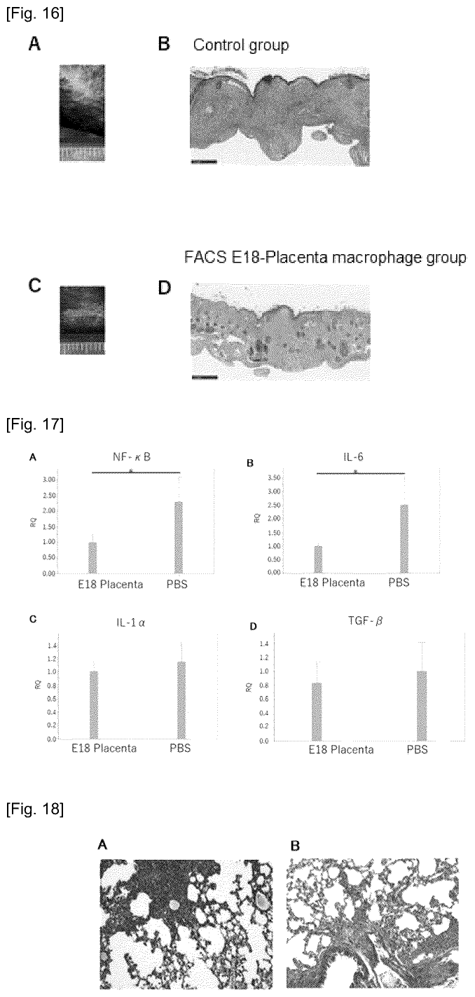

Real-time PCR on day 14 showed that NF-kB, an inflammatory marker, was decreased in the FACS E18-Placenta macrophage group (FIG. 17(A)). As for cytokines, IL-6 was decreased (FIG. 17(B)). There was a decreasing trend in IL1α and TGFβ, but no significant difference compared to the PBS group (FIGS. 17(C) and (D)). These results indicate that the administration of fetal macrophages suppressed inflammation. These results indicate that increase in the purity of E18-Placenta macrophages (from about 80% to 99%) by FACS has the effect of promoting wound healing.

### Example 10: Inhibition of Lung Fibrosis by Fetal Macrophages in a Model of Pulmonary Fibrosis

E18-Placenta macrophages were obtained from the placenta of GFP mice on day 18 of pregnancy under the same conditions as in Example 5.

Two groups of five 8- to 10-week-old male BL6 mice were sedated under isoflurane inhalation anesthesia and treated with 2 mg/kg of bleomycin via airway. 24 hours later, one group was injected with 0.1 ml of 2×10⁶ E18-Placenta macrophages suspended in PBS and the other group was injected with 0.1 ml PBS through the femoral vein in the inguinal region.

Fourteen days later, the animals were sedated under isoflurane inhalation anesthesia and sacrificed to death by dislocation of the cervical vertebra. Lungs from each group were collected and soaked in 4% PFA after observation of a lung weight. Some specimens were embedded in paraffin and tissues were observed.

### (Results)

Representative tissue sections were Grade 5 (FIG. 18(A)) in the PBS-treated group and Grade 3 (FIG. 18(B)) in the fetal macrophage-treated group. Intravenous administration to a model of pulmonary fibrosis inhibited lung fibrosis and inflammation.

### Example 11: Inhibitory Effects of Amnion-Derived Yolk Sac Macrophages and Placenta-Derived Fetal Macrophages on Aging and Inflammation

E12-Aminion macrophages were obtained from the amnion of GFP mice on day 12 of pregnancy under the same conditions as in Example 5. However, the stirring conditions using a thermostatic shaker/stirrer were 37° C., 45 minutes, and 750 rpm.

Next, E18-Placenta macrophages were obtained from the placenta of GFP mice on day 18 of pregnancy under the same conditions as in Example 5. The stirring conditions were 37° C., 60 minutes, and 750 rpm using a thermostatic shaker/stirrer.

Mice were divided into three groups, each group with three 8- to 10-week-old male BL6 mice and three 70-week-old male BL6 mice. The 70-week-old mice were anesthetized with isoflurane inhalation. One group was injected with 0.1 ml of E12-Aminion macrophages rich in 1×10⁶ amnion-derived day 12 fetal macrophages (yolk sac macrophages) suspended in PBS, and the other group was injected with 0.1 ml of E18-Placenta macrophages rich in 5×10⁶ placenta-derived embryonic day 18 fetal macrophages suspended in PBS, each through the femoral vein in the inguinal region.

Three months later, the animals were sedated under isoflurane inhalation anesthesia and sacrificed to death by dislocation of the cervical vertebra. The skin of each group was collected and homogenized in ISOGEN, then RNA was extracted and cDNA synthesized. The synthesized cDNA was measured by real-time PCR using a TaqMan probe.

### (Results)

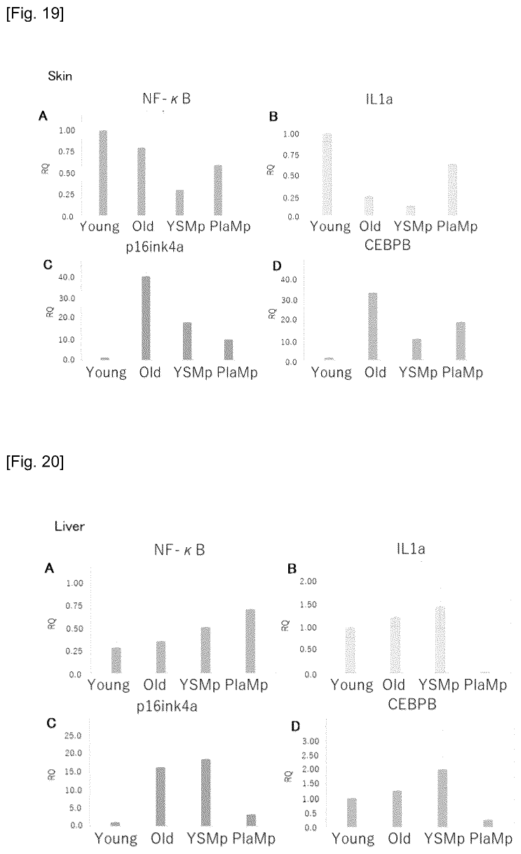

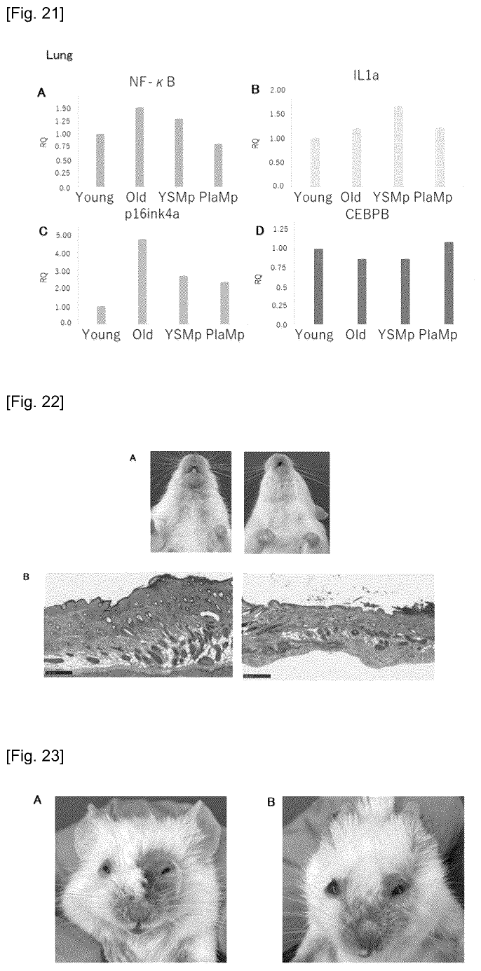

Skin aging markers in young mice and old mice were evaluated by PCR. FIGS. 19(A)-21(D) show changes in aging markers (p16ink4a, CEBPB), an inflammatory cytokine (IL-1α) and NF-KB in the skin of old mice when the skin of 8- to 10-week-old young mice was set as 1. The results show the effect of administration of yolk sac macrophages (Yolk Sac Macrophage) on embryonic day 12 and the effect of an intravenous administration of placental macrophages (Placenta Macrophage) on embryonic day 18 on aging mice to improve aging markers and suppress IL-1α and NFκB, which are indicators of inflammation.

### Example 12: Healing Effect of Fetal Macrophages on Atopic Dermatitis Model Mice (MACS)

A pellet containing cells of E18-Placenta macrophages were obtained from BALB/c mice on day 18 of pregnancy, by the same method described in Example 5. The E18-Placenta macrophages were subsequently obtained by MACS in the same manner as in Example 5.

### (Animal Experiments)

Six 11-week-old male ADMJ mice were used. Videos were taken regarding scratching behavior. The mice were then sedated under isoflurane inhalation anesthesia, one group was injected with 0.2 ml of the E18-Placenta macrophages 1×10⁶ collected by MACS and suspended in PBS and the other group was injected with 0.2 ml of PBS, respectively, via tail vein using a micro cannula. One dose administration each week was repeatedly four times, and a video of the scratching behavior observation was taken one week later. The mice were then sacrificed, skin tissue was collected, and tissue sections were observed with Masson trichrome staining.

### (Behavioral Analysis by ELAN)

Five weeks after the first injection, the scratching behavior was analyzed, and then the tissue was collected for histological observation. The scratching behavior was photographed from four directions to eliminate blind spots, and analyzed using ELAN (EUDICO Linguistic Annotator) software. In the ELAN analysis, the average of the number of scratches counted blind by four members was evaluated.

### (Results)

Gross observations showed a prominent improvement in the mandible area (FIG. 22(A)), although facial alopecia varied between the PBS group and E18-Placenta macrophage administration group. Histological findings showed an inflammatory cell infiltration and skin thickening in the PBS group, whereas a slight inhibition of epidermal thickening was observed in the E18-Placenta macrophage administration group (FIG. 22(B)).

In addition, in the case of administration of the E18-Placenta macrophages collected by FACS as in additional example 2, the treatment was remarkably effective. Half of the face was in a state of alopecia before treatment (FIG. 23 left), but showed clear improvement (FIG. 23 right).

### (Scratch Behavioral Analysis)

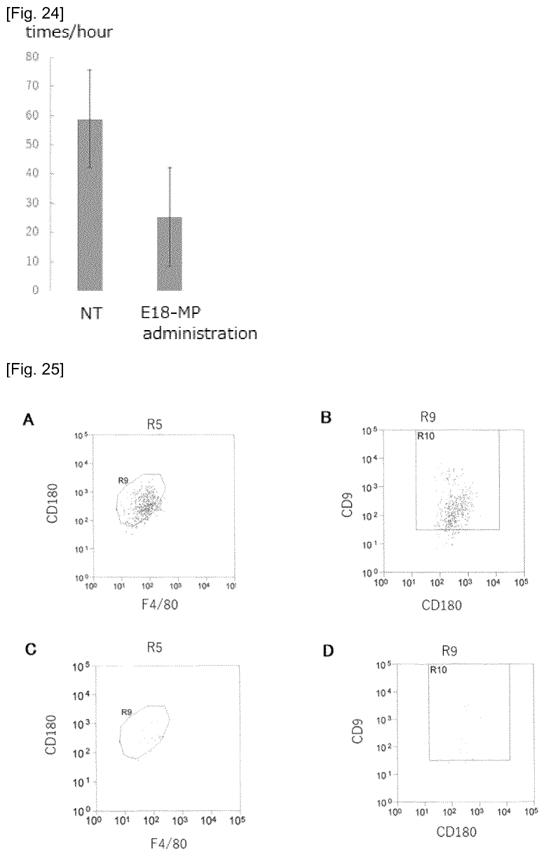

The results of ELAN analysis of the number of scratches in the behavior analysis video recording conducted before tissue collection showed that the number of scratches decreased in the group treated with fetal macrophages, although there was no significant difference (FIG. 24, Table 1).

TABLE 1

| Mouse | | | | | | |
|---|---|---|---|---|---|---|
| No. | −30 | −60 | −90 | −210 | Total 3.5 hours | |
| 1 | 44 | 141 | 93 | 3 | 281 | times |
| 2 | 8 | 68 | 17 | 7 | 100 | times |
| 3 | 17 | 60 | 77 | 83 | 237 | times |
| 4 | 10 | 7 | 32 | 14 | 63 | times |
| 5 | 3 | 19 | 26 | 20 | 68 | times |
| 6 | 52 | 30 | 36 | 16 | 134 | times |

* Mice No. 1 to 3: untreated group, No. 4 to 6: E-18 Placenta macrophage administration group

### Example 13: Differentiation and Adhesion of Fetal Macrophages

A pellet containing cells of E18-Placenta macrophages were obtained from ICR mice on day 18 of pregnancy by the same method described in Example 5. E18-Placenta macrophages were then obtained by FACS in the same manner as described in Example 9. The E18-Placenta macrophages collected by FACS were cultured.
(1) E18-Placenta macrophages collected by FACS were divided equally into the DMEM low glucose (FUJIFILM Wako Pure Chemicals Co., Ltd.) medium supplemented with 10% BSA and 1% PS and the DMEM/F12 (FUJIFILM Wako Pure Chemicals Co., Ltd.) medium supplemented with 10% BSA and 1% PS. Each medium was seeded into adhesive 6-well plates. 1 week later, the culture supernatant was collected (collected at 8.5×10⁴ cell/well in both cases) and again analyzed by FACS as in Example 9. It was collected in both cases.
(2) 1.2×10⁵ E18-Placenta macrophages collected by FACS were divided equally into the medium containing DMEM low glucose supplemented with heat-inactivated 10% BSA and 1% PS and medium containing DMEM low glucose supplemented with heat-inactivated 10% BSA, 1% PS and CSF1 (1 ng/ml). Each medium was then seeded into adherent 6-well plates. 1 week later, the culture supernatant was collected (collected at 6×10⁴ cells/well in both cases) and again analyzed by FACS as in Example 9.
(3) 1.4×10⁵ E18-Placenta macrophages collected by FACS were equally divided into the medium containing DMEM low glucose supplemented with heat-inactivated 10% BSA and 1% PS and the medium containing DMEM low glucose supplemented with heat-inactivated 10% BSA, 1% PS and CSF1 (1 ng/ml). Each medium was then seeded into non-adherent 6-well plates. 5 days later, the culture supernatant was collected (collected at 7×10⁴ cells/well in both cases) and again analyzed by FACS as in Example 9.

### (Results)

### (1) Comparison Between DMEM Low Glucose and DMEM/F12

Most of the cells in the group cultured in the DMEM low glucose supplemented with heat-inactivated 10% BSA and 1% PS were floating in the culture supernatant (FIGS. 25(A), (B)). The cells in this culture supernatant were CD180 and CD9 positive and retained the status of fetal macrophages. On the other hand, cells in the group cultured in DMEM/F12 supplemented with heat-inactivated 10% BSA and 1% PS were more differentiated and adhered to the plate, and fewer cells were floating in the culture supernatant (FIGS. 25(C), (D)).

(2) Comparison Between with CSF1 and without CSF1

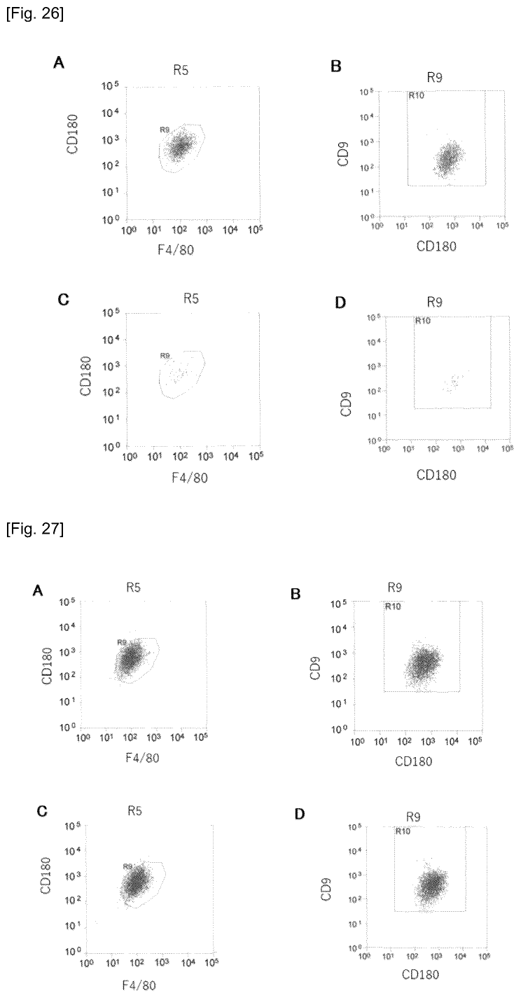

Most of the cells in the group cultured in DMEM low glucose supplemented with heat-inactivated 10% BSA and 1% PS were floating in the culture supernatant (FIGS. 26(A), (B)). Cells in the group cultured in DMEM low glucose supplemented with heat-inactivated 10% BSA, 1% PS and CSF1 (1 ng/ml) were more differentiated and attached to the plate, and fewer cells were suspended in the culture supernatant (FIGS. 26(C), (D)).

### (3) Effect of Using Non-Adherent Culture Dishes

As a result of using non-adherent culture dishes, cells in the group cultured in DMEM low glucose supplemented with heat-inactivated 10% BSA and 1% PS under the same culture conditions as (2) were floating in the culture supernatant (FIGS. 27(A), (B)). The cells cultured in the DMEM low glucose supplemented with heat-inactivated 10% BSA, 1% PS, and CSF1 (1 ng/ml) under the same culture conditions as in (2) were floating in the culture supernatant, CD 180 and CD9 positive, and maintained the state of fetal macrophages (FIGS. 27(C), (D)).

As a result, it was confirmed that fetal macrophages have acquired adhesive ability as differentiation progressed.

### Example 14: Differentiation and Adhesion of Fetal Macrophages

A pellet containing cells of E18-Placenta macrophages was obtained from ICR mice on the day 18 of pregnancy by the same method described in Example 5. A blocking buffer including an anti-CD16/32 antibody was added to the cell pellet on ice and mixed at 4° C. for 15 minutes. Then, the anti-CD11b antibody, the anti-F4/80 antibody, the anti-CD180 antibody, the anti-CD9 antibody, DAPI, and the anti-MHC class II antibody were added and incubated at 4° C. for 30 minutes. To remove excess antibodies, the FACS buffer was added, and the mixture were centrifuged again at 300×g for 5 minutes at 4° C. for separation to create a pellet of cells. After removing the supernatant, the FACS buffer was added again to the precipitated pellet and analyzed by flow cytometry. As a comparison, spleens of adult animals were harvested by the same method described in Example 5 to obtain a pellet containing macrophages. The pellets were analyzed by flow cytometry in the same manner as described above.

### (Results)

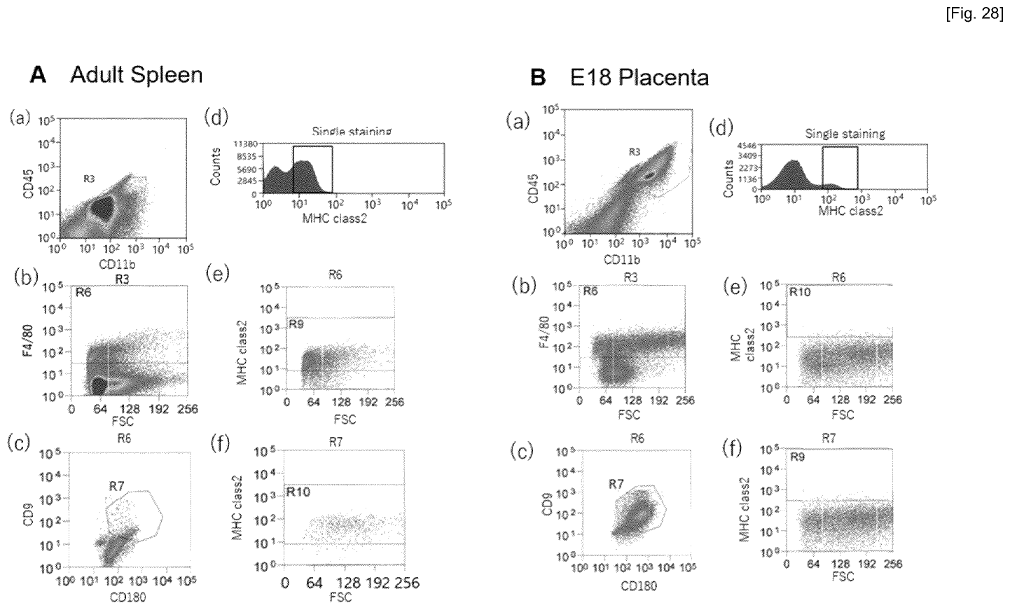

Compared with univariate histogram plots and adult animal spleens, the fetal macrophages of E18 placenta were negative for MHC class II (FIGS. 28A, B, especially FIG. 28B(d)). This proves that the fetal macrophages are immune tolerant.

1-16. (canceled)

17. A cellular therapeutic preparation comprising, as an active ingredient,
a cell population as an active ingredient comprising macrophages that are CD11b positive, F4/80 positive, CD180 positive, and CD9 positive and that are derived from a tissue selected from the group consisting of placenta, umbilical cord and amnion as an active ingredient with a percentage of 75% or more, the cell population having cell viability of 80% or more.

18. The cellular therapeutic preparation according to claim 17, wherein the macrophages are non-adherent cells.

19. The cellular therapeutic preparation according to claim 17, wherein the macrophages include Hofbauer cells.

20. The cellular therapeutic preparation according to claim 17, wherein the cellular therapeutic preparation is an injectable preparation.

21. A method of transplanting cells into a subject, comprising administering a therapeutically effective amount of the cellular therapeutic preparation according to claim 17 to the subject.

22. A method of treating a disease selected from the group consisting of an inflammatory disease, an immunomodulatory disease, and an autoimmune disease in a subject, comprising administering a therapeutically effective amount of the cellular therapeutic preparation according to claim 17 to the subject.

23. The method according to claim 22, wherein the inflammatory disease includes inflammatory bowel disease or chronic inflammatory disease.

24. The method according to claim 22, wherein the immunomodulatory disease includes a graft-versus-host disease, systemic lupus erythematosus, liver cirrhosis, pulmonary fibrosis, atopic dermatitis, or aging.

25. A method of treating a subject, comprising administering a therapeutically effective amount of the cellular therapeutic preparation according to claim 17 to the subject, wherein the treating include promotion of epithelialization, promotion of angiogenesis, inhibition of scarring, inhibition of thickening of the skin, suppression of wrinkle, healing of wound, or suppression of fibrosis.

26. A cellular therapeutic preparation comprising a cell population comprising macrophages that are CD11b positive, F4/80 positive, CD180 positive, and CD9 positive and that are derived from a tissue selected from the group consisting of placenta, umbilical cord and amnion as an active ingredient.

27. The cellular therapeutic preparation according to claim 26, wherein the macrophages are non-adherent cells.

28. The cellular therapeutic preparation according to claim 26, wherein the macrophages include Hofbauer cells.

29. The cellular therapeutic preparation according to claim 26, wherein the cellular therapeutic preparation is an injectable preparation.

30. A method of transplanting cells into a subject, comprising administering a therapeutically effective amount of the cellular therapeutic preparation according to claim 26 to the subject.

31. A method of treating a disease selected from the group consisting of an inflammatory disease, an immunomodulatory disease, and an autoimmune disease in a subject, comprising administering a therapeutically effective amount of the cellular therapeutic preparation according to claim 26 to the subject.

32. The method according to claim 31, wherein the inflammatory disease includes inflammatory bowel disease or chronic inflammatory disease.

33. The method according to claim 31, wherein the immunomodulatory disease includes a graft-versus-host disease, systemic lupus erythematosus, liver cirrhosis, pulmonary fibrosis, atopic dermatitis, or aging.

34. A method of treating a subject, comprising administering a therapeutically effective amount of the cellular therapeutic preparation according to claim 31 to the subject, wherein the treating include promotion of epithelialization, promotion of angiogenesis, inhibition of scarring, inhibition of thickening of the skin, suppression of wrinkle, healing of wound, or suppression of fibrosis.

35. A cosmetic composition comprising the cell population according to claim 17.

36. A cosmetic composition comprising the cell population according to claim 26.
